(19) United States
(12) Patent Application Publication
(10) Pub. No.: US 2026/0229321 A1
(43) Pub. Date: Aug. 6, 2026

(54) PREDICTION AND VERIFICATION SYSTEM FOR COVER WINDOW MANUFACTURING AND ELECTRONIC DEVICE INCLUDING THE SAME

(71) Applicant: SAMSUNG DISPLAY CO., LTD., YONGIN-SI (KR)

(72) Inventor: BEOMGYU CHOI, YONGIN-SI (KR)

(21) Appl. No.: 19/047,004

(22) Filed: Feb. 6, 2025

(30) Foreign Application Priority Data
Apr. 29, 2024 (KR) 10-2024-0057058

(51) Int. Cl. G16C 60/00; G16C 20/70

(52) U.S. Cl. CPC G16C 60/00; G16C 20/70

(57) ABSTRACT

A prediction and verification system is provided. The system includes a data processing unit that processes raw prediction data to generate processed prediction data, which includes viscosity data. This viscosity data is calculated considering the material properties of the window sheet material and the process temperature. Subsequently, a machine-learning unit receives this processed prediction data and predicts dimension values of a cover window, including the length, width, and height, which are collectively output as dimension data. A determination unit then evaluates this dimension data against predefined target data to ascertain the suitability of the raw prediction data for practical application in a manufacturing process for manufacturing the cover window.

### CROSS-REFERENCE TO RELATED APPLICATIONS

This U.S. non-provisional patent application claims priority under 35 U.S.C. § 119 to Korean Patent Application No. 10-2024-0057058, filed on Apr. 29, 2024, the disclosure of which is incorporated by reference herein.

### 1. TECHNICAL FIELD

The present disclosure is directed to a prediction and verification system for cover window manufacturing and an electronic device including the same.

### 2. DISCUSSION OF RELATED ART

A display panel such as a liquid crystal display (LCD), a plasma display panel (PDP), or a light emitting diode (LED) display panel is protected by a cover window installed on its front surface. The cover window protects the display panel from physical damage and environmental contaminants, which prolongs the device's lifespan. Additionally, it enhances the display's aesthetics and optical quality with treatments that reduce glare and improve visibility. For touch-enabled devices, the cover window may also integrate touch technology, allowing for direct user interaction with the display surface.

The cover window may be manufactured through a thermal molding process. The process may involve heating a glass or plastic material until it becomes pliable, then shaping it in a mold to achieve the desired dimensions and curvature.

However, manufacturing the cover window via thermal molding presents challenges such as material variation, where different materials react uniquely to heat, affecting the dimensions of the final product. Dimensional inaccuracies in the cover window can significantly impact the performance and aesthetics of display devices. When dimensions vary, even slightly, the cover window may not fit properly on the display panel, leading to gaps or overlaps that compromise the device's integrity and protection. Such discrepancies can also affect the uniformity of the display's touch sensitivity if the cover window is part of a touch screen. Moreover, dimensional mismatches can allow dust and moisture to enter the device, potentially damaging the electronic components inside and reducing the overall lifespan of the device.

### SUMMARY

At least one embodiment of the present disclosure provides a system for predicting dimensions of a cover window based on input data and verifying the dimensions (e.g., a prediction and verification system), and a device for manufacturing the cover window (e.g., a cover window manufacturing device) using the input data upon verifying the dimensions.

An embodiment of the inventive concept provides a prediction and verification system including, a data processing unit configured to process raw prediction data to generate processed prediction data, a machine-learning unit configured to receive the processed prediction data from the data processing unit, predict dimension values of a cover window based on the processed prediction data and output the dimension values as dimension data, and a determination unit configured to compare the dimension data with target data to verify suitability of the raw prediction data for use in a manufacturing process for manufacturing the cover window. The process prediction data includes viscosity data of a window sheet material, calculated based on a material of the window sheet material and a process temperature. The dimension data includes a length, a width, and a height of the cover window.

In an embodiment, the data processing unit calculates the viscosity data based additionally on first information about a condition of the manufacturing process for manufacturing the cover window, and generates the processed prediction data additionally based on second information about a mold used in the manufacturing process, third information about a shape of a cover window sample, and fourth information about a length and width of the window sheet material.

In an embodiment, the first information may include information about a process time of the manufacturing process, information about the process temperature in the manufacturing process, and information about a process pressure in the manufacturing process.

In an embodiment, the cover window is manufactured through a pre-heat process, a molding process, and a cooling process, wherein the pre-heat process includes a plurality of pre-heat processes, the molding process includes a plurality of molding processes, the cooling process includes a plurality of cooling processes, the process temperature includes temperatures in the plurality of pre-heat processes, temperatures in the plurality of molding processes, and temperatures in the plurality of cooling processes, and the process pressure includes one pressure in the plurality of pre-heat processes, and respective pressures in the plurality of molding processes.

In an embodiment, the viscosity data may include first viscosity data calculated using the temperatures in the plurality of pre-heat processes, second viscosity data calculated using the temperatures in the plurality of molding processes; and third viscosity data calculated using the temperatures in the plurality of cooling processes.

In an embodiment, the mold may include a lower mold including a molding part and an upper mold disposed on the lower mold, and the second information may include pieces of information about an interval between the lower mold and the cover window sample, a width of a molding part of the lower mold, an interval between the upper mold and the lower mold, and a curvature correction amount of the window sheet material.

In an embodiment, the fourth information may include information about a length and a width of the window sheet material.

In an embodiment, the fourth information may further include information about a residual, wherein the residual is calculated as a value obtained by subtracting one of the whole length or the whole width of the window sheet material from an average value of a length of the molding part of the upper mold and a length of the molding part of the lower mold.

In an embodiment, the third information may include information about a difference between a length of a top surface of the cover window sample and a length of a bottom surface of the cover window sample.

In an embodiment, the third information may further include information about a length of the cover window sample, a width of the cover window sample, and a height of the cover window sample.

In an embodiment, the third information may further include information about an angle of a bent portion of the cover window sample, a maximum curvature of the bent portion of the cover window sample, a bent width of the bent portion, and a curved shape of the cover window sample.

In an embodiment, the machine-learning unit may include a learning unit configured to receive sample input data from a dataset to perform training in a training process to output a trained result, and output the dimension data on the basis of the process prediction data in a prediction process, and a comparison unit configured to receive sample dimension data from the dataset and compare the output data with the sample dimension data.

In an embodiment, the learning unit may include an input layer including input nodes and configured to store the processed prediction data received from the data processing unit in the input nodes, a hidden layer including a plurality of sub-hidden layers, wherein sub-nodes of a first sub-hidden layer among the sub-hidden layers are connected to the input nodes, and an output layer including output nodes connected to sub-nodes of a last sub-hidden layer among the sub-layers, and configured to output result values stored in the output nodes as the dimension data.

In an embodiment, the result values stored in the output nodes are pieces of information about the length, the width, and the height of the cover window.

In an embodiment of the inventive concept, a cover window manufacturing device includes a dimension prediction system configured to predict dimension values of a cover window for generating dimension data, and a window molding device configured to mold a window sheet material to manufacture the cover window when the dimension data matches target data, wherein the dimension prediction system includes a data processing unit configured to process the raw prediction data to generate processed prediction data, a machine-learning unit configured to receive the processed prediction data from the data processing unit, predict the dimension values based on the processed prediction data to output the dimension values as the dimension data, and a determination unit configured to compare the dimension data with the target data to verify suitability of the raw prediction data for use in a manufacturing process for manufacturing the cover window. The raw prediction data includes viscosity data of the window sheet material, calculated based on a material of the window sheet material and a process temperature.

In an embodiment, the data processing unit calculates the viscosity data additionally based on first information about a condition of the manufacturing process for manufacturing the cover window, and generates the processed prediction data additionally based on second information about a mold used in the manufacturing process, third information about a shape of a cover window sample, and fourth information about a length and width of the window sheet material.

In an embodiment, the first information may include information about a process time of the manufacturing process, information about the process temperature in the manufacturing process, and information about a process pressure in the manufacturing process.

In an embodiment, the cover window may be manufactured through a pre-heat process, a molding process, and a cooling process, wherein the pre-heat process includes a plurality of pre-heat processes, the molding process includes a plurality of molding processes, the cooling process includes a plurality of cooling processes, the process temperature includes temperatures in the plurality of pre-heat processes, temperatures in the plurality of molding processes, and temperatures in the plurality of cooling processes, and the process pressure includes one pressure in the plurality of pre-heat processes, and respective pressures in the plurality of molding processes.

In an embodiment, the viscosity data may include first viscosity data calculated using the temperatures in the plurality of pre-heat processes, second viscosity data calculated using the temperatures in the plurality of molding processes, and third viscosity data calculated using the temperatures in the plurality of cooling processes.

In an embodiment of the inventive concept, a system for manufacturing a cover window includes: a data processing unit configured to process raw prediction data to generate processed prediction data, wherein the processed prediction data comprises viscosity data calculated based on a material of a window sheet material and a process temperature; a machine-learning unit configured to receive the processed prediction data from the data processing unit, predict dimension values of the cover window based on the processed prediction data and output the dimension values as dimension data, wherein the dimension data includes a length, a width, and a height of the cover window; a determination unit configured to compare the dimension data with target data to verify suitability of the raw prediction data for use in a manufacturing process for manufacturing the cover window; and a window molding device configured to mold the window sheet material into the cover window based on the raw prediction data when the determination unit verifies the raw prediction data.

### BRIEF DESCRIPTION OF THE FIGURES

The above features of the present disclosure will become readily apparent by reference to the following detailed description when considered in conjunction with the accompanying drawings wherein:

FIG. 1A is a block diagram showing a device for manufacturing a cover window (e.g., a cover window manufacturing device) according to an embodiment of the inventive concept;

FIG. 1B is a perspective view of the cover window according to an embodiment of the inventive concept;

FIG. 2A is a block diagram showing a machine-learning unit for predicting dimensions of the cover window according to an embodiment of the inventive concept;

FIG. 2B shows a learning unit of the machine-learning unit according to an embodiment of the inventive concept;

FIG. 3A is a cross-sectional view of a window molding device according to an embodiment of the inventive concept;

FIG. 3B is a cross-sectional view showing a state in which a window sheet material is provided to the window molding device shown in FIG. 3A;

FIG. 4A is a block diagram showing a process for manufacturing a cover window according to an embodiment of the inventive concept;

FIG. 4B is a block diagram showing a pre-heat process according to an embodiment of the inventive concept;

FIG. 4C is a block diagram showing a molding process according to an embodiment of the inventive concept;

FIG. 4D is a block diagram showing a cooling process according to an embodiment of the inventive concept;

FIG. 5 is a graph showing the relationship between a temperature and a viscosity according to an embodiment of the inventive concept;

FIG. 6 is a table showing viscosity versus temperature for each window sheet material according to an embodiment of the inventive concept;

FIG. 7A is a perspective view of the window sheet material according to an embodiment of the inventive concept;

FIG. 7B is a cross-sectional view of the window sheet material cut along A-A′ of FIG. 7A;

FIG. 8A is a cross-sectional view of the window molding device according to an embodiment of the inventive concept;

FIG. 8B is an enlarged cross-sectional view of a partial area of FIG. 8B;

FIG. 9 is a perspective view of a cover window sample shown in FIG. 8A;

FIG. 10 is a cross-sectional view of the cover window sample cut along B-B′ of FIG. 9;

FIG. 11 is an enlarged cross-sectional view of a partial area of FIG. 10;

FIG. 12 is a cross-sectional view of the cover window sample cut along C-C′ of FIG. 9; and

FIGS. 13A and 13B are cross-sectional views showing the shapes of cover window samples according to embodiments of the inventive concept.

### DETAILED DESCRIPTION

It will be understood that when an element or layer is referred to as being “on”, “connected to” or “coupled to” another element or layer, it can be directly on, connected or coupled to the other element or intervening third elements may be present. Like reference numerals in the drawings refer to like elements. The term “and/or” includes all combinations of one or more of which associated configurations may define.

Terms such as first, second, and the like may be used to describe various elements, but these elements should not be limited by the terms. Such terms are only used for distinguishing one element from other elements. For instance, a first component may be referred to as a second component, or similarly, a second component may be referred to as a first component, without departing from the scope of the present disclosure. The singular expressions include plural expressions unless the context clearly dictates otherwise.

In addition, the terms such as “under”, “lower”, “on”, and “upper” are used for explaining associations of items illustrated in the drawings. The terms are used as a relative concept and are described with reference to the direction indicated in the drawings.

It should be understood that the terms “comprise” or “have” are intended to specify the presence of stated features, integers, steps, operations, elements, components, or combinations thereof in the disclosure, but do not preclude the presence or addition of one or more other features, integers, steps, operations, elements, components, or combinations thereof.

Hereinafter, embodiments of the inventive concept will be described with reference to the accompanying drawings.

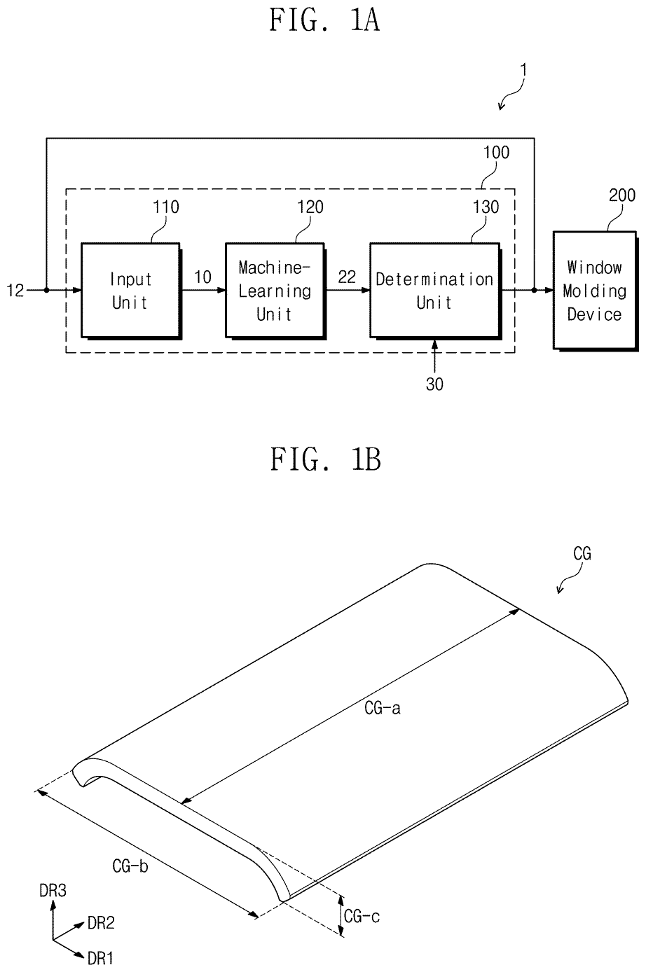

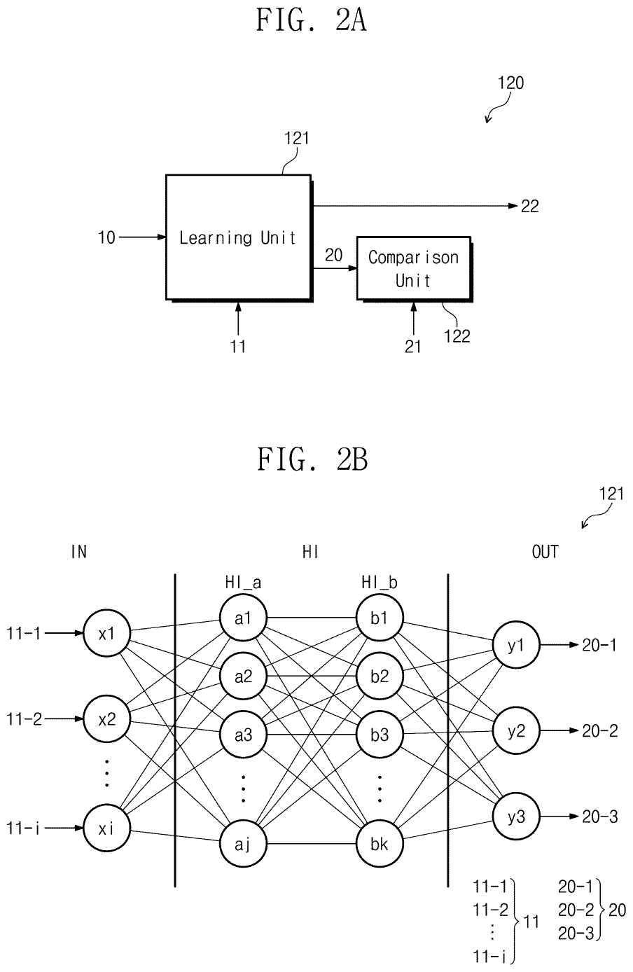

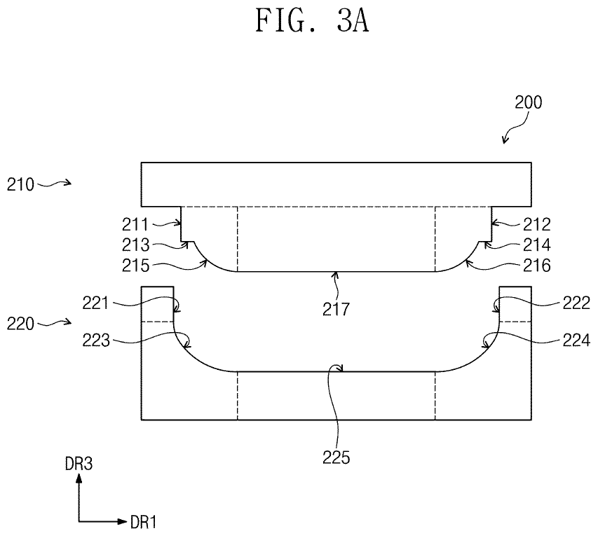

FIG. 1A is a block diagram showing a device for manufacturing a cover window (e.g., a cover window manufacturing device) according to an embodiment of the inventive concept. FIG. 1B is a perspective view of the cover window according to an embodiment of the inventive concept. FIG. 2A is a block diagram showing a machine-learning unit for predicting dimensions of the cover window according to an embodiment of the inventive concept. FIG. 2B shows a learning unit of the machine-learning unit according to an embodiment of the inventive concept.

Referring to FIG. 1A, the cover window manufacturing device 1 may include a system 100 for predicting and verifying dimensions of a cover window (e.g., dimension prediction system or a prediction and verification system) and a window molding device 200.

The dimension prediction system 100 is a system for predicting dimension values of a cover window CG (see FIG. 1b) and may include an input unit 110 (e.g., a data processing unit), a machine-learning unit 120, and a determination unit 130. The input unit 110, the machine-learning unit 120, and the determination unit 130 may each be implemented by software or hardware. For example, the units may be computer programs executed by a processor, or individual processors or logic circuits or a combination thereof.

The input unit 110 may provide data required to predict the dimension values of the cover window CG to the machine-learning unit 120 as input data 10. The input data 10 may include information (hereinafter, referred to as first information) about conditions of a manufacturing process for the cover window CG and information (hereinafter, referred to as second information) about a mold used in the manufacturing process. The mold may be a precision-engineered tool designed to shape window sheet material into the specific dimensions and contours required for the cover window CG. The mold may include a cavity and a core. The cavity is the part of the mold that shapes the window sheet material into the desired form and the core fits into the cavity and shapes features like holes on the interior of the molded part. The input data 10 may further include information (hereinafter, referred to as third information) about the shape of a cover window sample and information (hereinafter, referred to as fourth information) about the length and width of the window sheet material used for the cover window CG.

The input unit 110 may receive, as prediction input data 12 (e.g., raw prediction data) from a user, information about the cover window CG intended to predict, the manufacturing process conditions, and information about the window sheet material. The input unit 110 may process the prediction input data 12 to generate the input data 10 (e.g., processed prediction data). As an example of the inventive concept, the input data 10 may include viscosity data. The input unit 110 may use the information about the manufacturing conditions (e.g., information about process temperatures) and information about the window sheet material to calculate the viscosity data.

The machine-learning unit 120 may receive the input data 10 from the input unit 110. The machine-learning unit 120 may analyze and learn from the input data 10 through a machine-learning algorithm to predict the dimension values of the cover window CG. The machine-learning algorithm is a process in which a computer analyzes and learns data to cause the computer to perform specific tasks. The machine-learning algorithm may include an algorithm of regression analysis (ElasticNet), partial least squares, extreme gradient boosting (XGBOOST), or deep neural network (DNN).

Referring to FIGS. 2A and 2B, the machine-learning unit 120 may include a learning unit 121 and a comparison unit 122. The units may be implemented by software, hardware (e.g., processors, logic circuits, etc.) or a combination thereof.

The learning unit 121 may receive the input data 10 or sample input data 11. The learning unit 121 may perform a training process and a prediction process. In the training process, the learning unit 121 receives the sample input data 11 from a dataset, and not only calculates output data 20 but also updates its internal parameters using the sample input data 11 to enhance prediction accuracy. In the prediction process, the learning unit 121 receives the input data 10 from the input unit 110 and uses the internal parameters (e.g., weights) adjusted during the prior training to output dimension data 22, which is the predicted dimensions based on the input data 10. FIG. 2B shows a case where the learning unit 121 receives the sample input data 11 to output the output data 20, but the learning unit 121 may also receive the input data 10 to output the dimension data 22.

The learning unit 121 may include an input layer IN, a hidden layer HI, and an output layer OUT.

The input layer IN may include a plurality of input nodes (namely, a first input node x1, and a second input node x2 to an i-th input node xi). The input layer IN may be provided with the sample input data 11 from the input unit 110. The sample input data 11 may include i (i is a positive integer) pieces of data (namely, first input data 11-1, and second input data 11-2 to i-th input data 11-i). The first to i-th pieces of input data 11-1 to 11-i may be respectively input to the first to i-th input nodes (x1 to xi) of the input layer IN.

The hidden layer HI may include a first sub-hidden layer HI_a and a second sub-hidden layer HI_b. In FIG. 2B, a structure in which the hidden layer HI includes two sub-hidden layers HI_a and HI_b is shown, but the hidden layer HI may have three or more sub-hidden layers. The first sub-hidden layer HI_a may include j (is a positive integer) first sub-nodes a1, a2, and a3 to aj. Each input node x1 to xi of the input layer IN may be connected to the j first sub-nodes a1 to aj.

Different weights may be respectively set between the input nodes x1 to xi and the j first sub-nodes a1 to aj. A first weight is set between the first input node x1 and a (1-1)-th node a1 among the j first sub-nodes a1 to aj, and a second weight is set between the first input node x1 and a (1-2)-th node a2 among the j first sub-nodes a1 to aj. The first weight may be different from the second weight.

As the i input nodes x1 to xi are connected to the j first sub-nodes a1 to aj, the number of weights between the input layer IN and the first sub-hidden layer HI_a may be defined as i×j. The second sub-hidden layer HI_b may include k (k is a positive integer) second sub-nodes b1 to bk. Each of the first sub-nodes a1 to aj of the first sub-hidden layer HI_a may be connected to the k second sub-nodes b1 to bk. Accordingly, as the j first sub-nodes a1 to aj are connected to the k second sub-nodes b1 to bk, the number of the weights between the first sub-hidden layer HI_a and the second sub-hidden layer HI_b may be defined as j×k.

When the hidden layer HI includes n (n is a positive integer) sub-hidden layers, each (n−1)-th sub-node of the (n−1)-th sub-hidden layer may be connected to n-th sub-nodes of the n-th sub-hidden layer. Accordingly, the number of weights between the (n−1)-th hidden layer and the n-th sub-hidden layer may be (the number of (n−1)-th sub-nodes)×(the number of n-th sub-nodes).

The first to i-th pieces of input data 11-1 to 11-i respectively stored in the input nodes x1 to xi of the input layer IN are multiplied by the weights set between the input nodes x1 to xi and the (1-1)-th node a1 to calculate a weighted input value for each of the input nodes x1 to xi. The sum of the weighted input values through each of the input nodes x1 to xi may be stored in the (1-1)-th node of the first sub-hidden layer HI_a. The sum may be limited to a value in a range preset through a non-linear process. The non-linear process may be performed by means of the sigmoid function, the ReLu function, the hyperbolic tangent function, or the like. Similarly, the result values obtained through the above-described process may also be respectively stored in the remaining nodes a2 to aj of the first sub-nodes a1 to aj, namely, the (1-2)-th node a2 to (1-j)-th node aj.

When the first sub-nodes a1 to aj of the first sub-hidden layer HI_a have the result values obtained through the above-described process, the respective first sub-node values of the first sub-nodes a1 to aj may be calculated by multiplying the result values respectively stored in the first sub-nodes a1 to aj of the first sub-hidden layer HI_a by the weights set between the first sub-nodes a1 to aj and a (2-1)-th node b1. The sum value of the first sub-node values may be stored in the (2-1)-th node b1 of the second sub-hidden layer HI_b. The sum value may be limited to a value in a range preset through the non-linear process. Similarly, the result values obtained through the above-described process may also be respectively stored in the remaining nodes b2 to bk of the second sub-nodes b1 to bk, namely, the (2-2)-th node b2 to (2-k)-th node bk.

The output layer OUT may include three output nodes y1 to y3. Each of the output nodes y1 to y3 of the output layer OUT may be connected to the second sub-nodes b1 to bk of the second sub-hidden layer HI_b. When the hidden layer HI includes n sub-hidden layers, each of the output nodes y1 to y3 of the output layer OUT may be connected to an n-th sub-node of an n-th sub-hidden layer. In FIG. 2B, a structure in which the output layer OUT includes the three output nodes y1 to y3 is shown, but when the number of dimensions intended to predict is m, the output layer OUT may include m output nodes y1 to ym. Here, m may be equal to or smaller than n.

Each of the output nodes y1 to y3 of the output layer OUT may also store result values acquired through an operation similar to the operations for storing values in the first sub-hidden layer HI_a and the second sub-hidden layer HI_b. The result values stored in the output nodes y1 to y3 may be the output data 20 predicted through the learning unit 121. As an example of the inventive concept, the result value stored in the first output node y1 among the output nodes y1 to y3 may include information about the whole length of the cover window CG, the result value stored in the second output node y2 may include information about the whole width of the cover window CG, and the result value stored in the third output node y3 may include information about the height of the cover window CG.

The comparison unit 122 receives the output data 20 from the learning unit 121 and receives sample dimension data 21 from the dataset. The comparison unit 122 compares the output data 20 with the sample dimension data 21. The sample dimension data 21 may be the dimension values of a sample cover window manufactured using information included in the sample input data 11.

The predicted dimensions of the sample cover window may be output as the output data 20. The output data 20 and the sample dimension data 21 may be compared with each other. According to a level of difference between the output data 20 and the sample dimension data 21, the weights between the input nodes x1 to xi of the input layer IN and the first sub-nodes a1 to aj of the first sub-hidden layer HI_a, the weights between the first sub-nodes a1 to aj of the first sub-hidden layer HI_a and the second sub-nodes b1 to bk of the second sub-hidden layer HI_b, and the weights between the second sub-nodes b1 to bk of the second sub-hidden layer HI_b and the output nodes y1 to y3 of the output layer OUT may be updated. As the number of weight updates increases, the difference between the output data 20 and the sample dimension data 21 may decrease.

The level of difference between the output data 20 and the sample dimension data 21 may be defined as a loss function. This means that as the value of the loss function decreases, the difference between the output data 20 and the sample dimension data 21 becomes smaller, and the weights are updated to reduce the value of the loss function. The loss function may include the mean square error (MSE) function, the root means squared error (RMSE) function, the mean absolute error (MAE) function, or the like.

As an example of the inventive concept, the number of weight updates may be limited to a preset reference number.

Similarly to the process in which the learning unit 121 receives the sample input data 11 to output the output data 20, the learning unit 121 may output the dimension data 22 when receiving the input data 10. In this case, the weights updated through the training process may be used in a process for calculating the dimension data 22.

Referring to FIGS. 1A and 1B, the determination unit 130 may receive the dimension values of the cover window CG, predicted by the machine-learning unit 120, as the dimension data 22. The determination unit 130 may receive desired target values of the cover window CG as target data 30.

The cover window CG may have the whole length CG-a, the whole width CG-b, and the height CG-c. The dimension data 22 and the target data 30 may include information about the whole length CG-a, the whole width CG-b, and the height CG-b of the cover window CG. The height CG-c may differ from the thickness of the cover window CG when the cover window CG is curved as shown in FIG. 1B. For example, the height CG-c may be the maximum vertical distance from the lowest point on a bottom surface to a highest point on an upper surface of the cover window CG.

The determination unit 130 may compare the dimension data 22 with the target data 30 to determine whether they are equal to one another (e.g., matches), and output a result of this comparison as a compared result. When the dimension data 22 is equal to the target data 30, the determination unit 130 may provide prediction input data 12 to the window molding device 200. Alternatively, when an error between the dimension data 22 and the target data 30 is within a certain range, the determination unit 130 may provide the prediction input data 12 to the window molding device 200. The determination unit 130 may verify suitability of the prediction input data 12 (e.g., raw prediction data) for use in a manufacturing process for manufacturing the cover window CG. Thus, the system 100 predicts whether the window molding device 200 is likely to manufacture a cover window CG with appropriate dimensions using the prediction input data 12. In an embodiment, when the dimension data 22 is not similar to the target data 30 (e.g., not equal to or within the certain range), the determination unit 130 does not provide the prediction input data 12 to the window molding device 200 to prevent it from manufacturing a cover window or provides an error signal to the window molding device 200. While FIG. 1A shows a signal line always providing the prediction input data 12 to the window molding device 200, this is not always the case. When the system 100 is being operated, the system 100 or the determination unit 130 may conditionally provide the prediction input data 12 to the window molding device 200 based on whether it has determined that the prediction input data 12 is likely to produce a cover window CG of appropriate dimensions. When the system 100 is not being operated or is powered down, the prediction input data 12 may be directly provided to the window molding device 200 through the signal line.

The window molding device 200 may manufacture the cover window CG using the prediction input data 12. The window molding device 200 may be used in the manufacturing process of the cover window CG, and the manufacturing process may include heat molding processes.

As described above, the learning unit 121 is trained through the sample input data 11, and the dimensions of the cover window CG may be predicted using the trained model. Accordingly, without a process for actually manufacturing the cover window, the dimensions of the cover window CG may be predicted through the trained model and the cover window having the desired target dimensions may be manufactured.

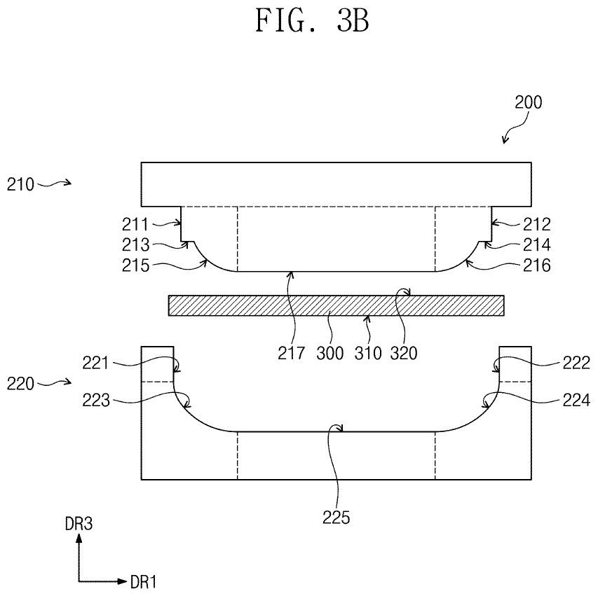

FIG. 3A is a cross-sectional view of the window molding device according to an embodiment of the inventive concept. FIG. 3B is a cross-sectional view showing a state in which a window sheet material is provided to the window molding device shown in FIG. 3A.

Referring to FIG. 3A, the window molding device 200 may include an upper mold 210 and a lower mold 220. The upper mold 210 and the lower mold 220 may be spaced apart in the third direction DR3.

The upper mold 210 may include a molding part. The molding part of the upper mold 210 may include a (1-1)-th upper portion 211, a (1-2)-th upper portion 212, a (2-1)-th upper portion 213, a (2-2)-th upper portion 214, a (3-1)-th upper portion 215, a (3-2)-th upper portion 216, and a fourth upper portion 217. The (3-1)-th upper portion 215 and the (3-2)-th upper portion 216 may include curved surfaces.

The lower mold 220 may include a molding part. The molding part of the lower mold 220 may include a (1-1)-th lower portion 221, a (1-2)-th lower portion 222, a (2-1)-th lower portion 223, a (2-2)-th lower portion 224, and a third lower portion 225. The (2-1)-th lower portion 223 and the (2-2)-th lower portion 224 may include curved surfaces.

Each of the upper mold 210 and the lower mold 220 may be composed of a metal material. However, the embodiment is not limited thereto, and each of the upper mold 210 and the lower mold 220 may be composed of a material such as ceramic, tungsten carbide (WC), silicon carbide (SiC), or the like.

Referring to FIG. 3B, the window sheet material 300 may be provided between the upper mold 210 and the lower mold 220.

The upper mold 210 may be used for pressurizing the window sheet material 300 to have curved surfaces. The upper mold 210 may be disposed on the window sheet material 300, and be moved toward a lower mold 220 to pressurize the window sheet material. When the upper mold 210 is moved toward the lower mold 220 to pressurize the window sheet material 300, the molding part of the upper mold 210 contacts the bottom surface 320 of the window sheet material 300 and the molding part of the lower mold 220 may contact the top surface 310 of the window sheet material 300.

When the window sheet material 300 is molded by the window molding device 200, the window sheet material 300 may be machined into the cover window CG having the curved surfaces in the third direction DR3. The bottom surface 320 of the window sheet material 300 may be molded into the curved surface in a portion in which the bottom surface 320 of the window sheet material 300 overlaps with the (3-1)-th upper portion 215 and the (3-2)-th upper portion 216 of the upper mold 210. The top surface 310 of the window sheet material 300 may be molded into the curved surface in a portion in which the top surface 310 of the window sheet material 300 overlaps with the (2-1)-th lower portion 223 and the (2-2)-th lower portion 224 of the lower mold 220.

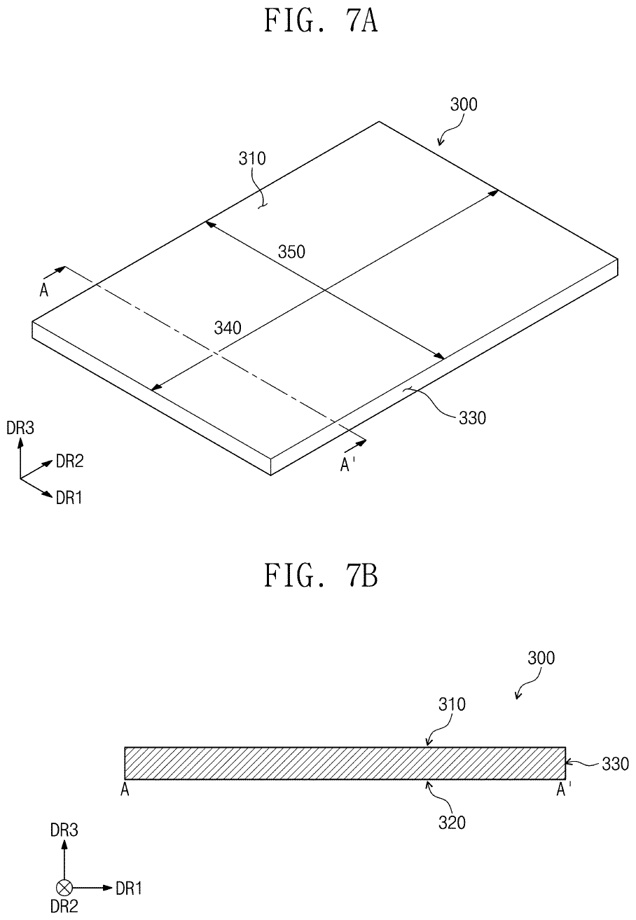

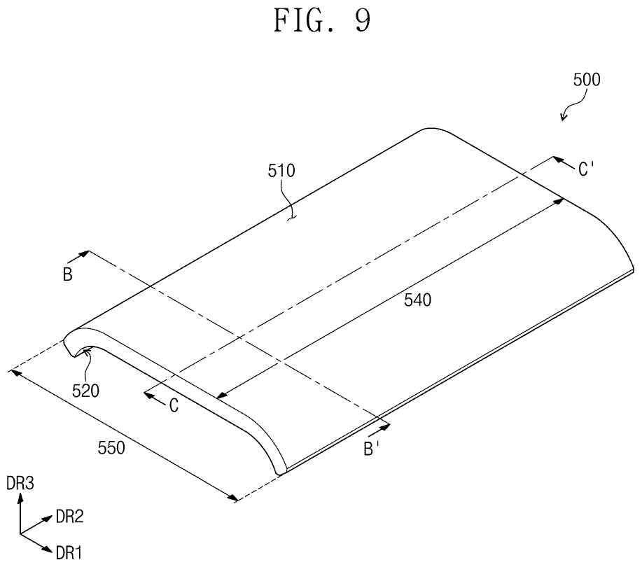

The input data 10 may include information (namely, the fourth information) about the length and width of the window sheet material 300. The fourth information may include information about a first residual and information about a second residual. The first residual may be calculated as a value obtained by subtracting the whole length 340 (see FIG. 7A) of the window sheet material 300 from the average value of the total length of the molding part of the upper mold 210 and the total length of the molding part of the lower mold 220. The second residual may be calculated as a value obtained by subtracting the whole width 350 (see FIG. 7A) of the window sheet material 300 from the average value of the total width of the molding part of the upper mold 210 and the total width of the molding part of the lower mold 220. The input unit 110 may determine, through the information about the first and second residuals, how different the whole length 340 and whole width 350 of the window sheet material 300 are in comparison to the molding parts of the upper mold 210 and the lower mold 220.

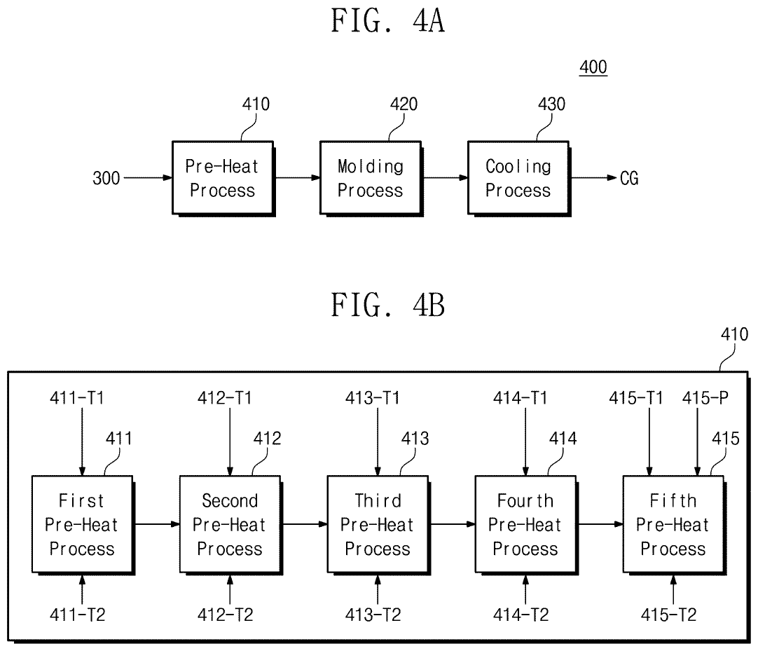

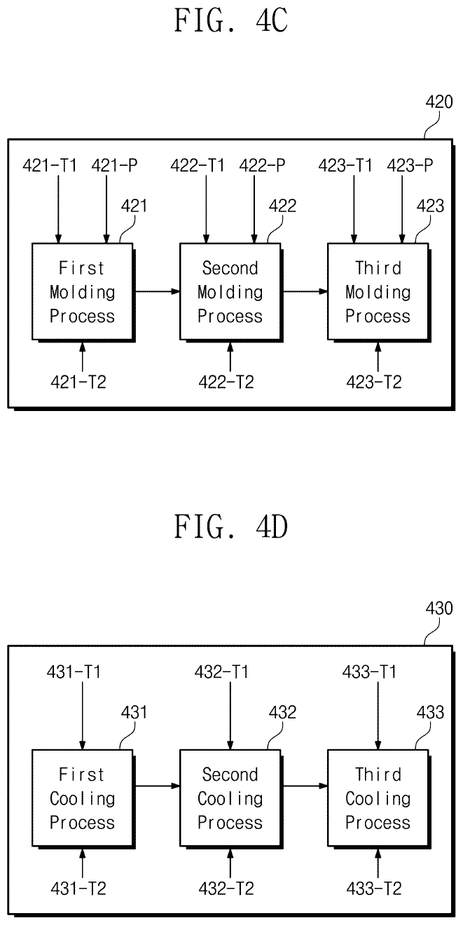

FIG. 4A is a block diagram showing a process of manufacturing a cover window (e.g., a cover window manufacturing process) according to an embodiment of the inventive concept. FIG. 4B is a block diagram showing a pre-heat process according to an embodiment of the inventive concept. FIG. 4C is a block diagram showing a molding process according to an embodiment of the inventive concept. FIG. 4D is a block diagram showing a cooling process according to an embodiment of the inventive concept.

Referring to FIGS. 4A to 4D, the manufacturing process of the cover window 400 according to an embodiment of the inventive concept includes a pre-heat process 410, a molding process 420, and a cooling process 430.

The window sheet material 300 (see FIG. 3B) disposed between the upper mold 210 (see FIG. 3B) and the lower mold 220 (see FIG. 3B) is heated in the pre-heat process 410, and the window sheet material 300 may be fluidized. The pre-heat process 410 may include first to fifth pre-heat processes 411 to 415. FIG. 4B illustrates that the pre-heat process 410 includes the first to fifth pre-heat processes 411 to 415, but a portion of the first to fifth pre-heat processes 411 to 415 may be omitted, or the pre-heat process 410 may further include an additional pre-heat process other than the first to fifth pre-heat processes 411 to 415. The first to fifth pre-heat processes 411 to 415 may be divided according to a temperature. For example, the second pre-heat process 412 may be performed at a higher temperature than the first pre-heat process 411, and the third pre-heat process 413 may be performed at a higher temperature than the second pre-heat process 412. Like this, the first to fifth pre-heat processes 411 to 415 may be divided according to a process temperature. As an example of the inventive concept, the pre-heat process 410 is performed in a manner where the process temperature is gradually increased in the first pre-heat process 411 to the fifth pre-heat process 415, but is not limited thereto. For example, the next pre-heat process may be performed at a temperature lower than that in the previous pre-heat process.

The first to fifth pre-heat processes 411 to 415 may be respectively performed at the first to fifth pre-heat process temperatures. The first to fifth pre-heat process temperatures may include first to fifth upper mold pre-heat temperatures 411-T1 to 415-T1 and first to fifth lower mold pre-heat temperatures 411-T2 to 415-T2. The first to fifth upper mold pre-heat temperatures 411-T1 to 415-T1 respectively indicate the temperatures of the upper mold 210 in the first to fifth pre-heat processes 411 to 415, and the first to fifth lower mold pre-heat temperatures 411-T2 to 415-T2 respectively indicate the temperatures of the lower mold 220 in the first to fifth pre-heat processes 411 to 415. Accordingly, the first to fifth pre-heat processes 411 to 415 may be performed in a state where the upper mold 210 is heated to the first to fifth upper mold pre-heat temperatures 411-T1 to 415-T1 and the lower mold 220 is heated to the first to fifth lower mold pre-heat temperatures 411-T2 to 415-T2.

The prediction input data 12 includes information (namely, the first information) about conditions of the manufacturing process for manufacturing the cover window CG. The first information may include information about the first to fifth upper mold pre-heat temperatures 411-T1 to 415-T1 and information about the first to fifth lower mold pre-heat temperatures 411-T2 to 415-T2.

In one pre-heat process among the first to fifth pre-heat processes 411 to 415, a heater disposed on the upper mold 210 of the window molding device 200 may press the upper mold 210. The heater may be part of a larger assembly that includes a mechanical actuator or press. In this case, the heater heats the upper mold 210, while the integrated actuator applies pressure to press the upper mold 210 against the window sheet material 300. For example, a fifth pre-heat pressure 415-P may be applied to the upper mold 210 in the fifth pre-heat process 415. However, the inventive concept is not limited thereto, and pressures may be applied to the upper mold 210 in at least two processes among the first to fifth pre-heat processes 411 to 415. Alternatively, the upper mold 210 is not pressed in any pre-heat process among the first to fifth pre-heat processes 411 to 415.

The first information may further include information about the fifth pre-heat pressure 415-P. However, the inventive concept is not limited thereto, and when there is a process in which an additional pressure is applied even in the first to fourth pre-heat processes 411 to 414, the first information may further include pressure information about each of the first to fourth pre-heat processes 411 to 414. When a pressure is not applied to the first to fifth pre-heat processes 411 to 415, the first information need not include the pressure information about each of the first to fifth pre-heat processes 411 to 415.

In the molding process 420, the window sheet material 300 (See FIG. 3B) may be machined to a curved shape by the window molding device 200 (See FIG. 3B). The molding process 420 may include first to third molding processes 421 to 423. FIG. 4C illustrates that the molding process 420 includes the first to third molding processes 421 to 423, but a portion among the first to third molding processes 421 to 423 may be omitted, or the molding process 420 may further include an additional molding process other than the first to third molding processes 421 to 423. The first to third molding processes 421 to 423 may be divided according to a temperature. For example, the second molding process 422 may be performed at a higher temperature than the first molding process 421, and the third molding process 423 may be performed at a higher temperature than the second molding process 422. As an example of the inventive concept, the molding process 420 is performed in a manner where the process temperature is gradually increased in the first molding process 421 to the third molding process 423, but is not limited thereto. For example, the next molding process may be performed at a temperature lower than that in the previous molding process. Alternatively, the first to third molding processes 421 to 423 may be divided according to a process pressure.

The first to third molding processes 421 to 423 may be respectively performed at the first to third molding process temperatures. The first to third molding process temperatures may include first to third upper mold molding temperatures 421-T1 to 423-T1 and first to third lower mold molding temperatures 421-T2 to 423-T2. The first to third upper mold molding temperatures 421-T1 to 423-T1 respectively indicate the temperatures of the upper mold 210 in the first to third molding processes 421 to 423, and the first to third lower mold molding temperatures 421-T2 to 423-T2 respectively indicate the temperatures of the lower mold 220 in the first to third molding processes 421 to 423. Accordingly, the first to third molding processes 421 to 423 may be performed in a state where the upper mold 210 is heated to the first to third upper mold molding temperatures 421-T1 to 423-T1 and the lower mold 220 is heated to the first to third lower mold molding temperatures 421-T2 to 423-T2.

The first information in the prediction input data 12 may include information about the first to third upper mold molding temperatures 421-T1 to 423-T1 and information about the first to third lower mold molding temperatures 421-T2 to 423-T2.

The first to third molding processes 421 to 423 may be respectively performed in states where first to third molding pressures 421-P to 423-P are applied. A heater disposed on the upper mold 210 of the window molding device 200 may apply a pressure to the upper mold 210.

The first information of the input data 10 may further include the first to third molding pressures 421-P to 423-P. However, the inventive concept is not limited thereto, and when there is a process in which a pressure is not applied among the first to third molding processes 421 to 423, the first information need not include pressure information about each of the first to third molding processes 421 to 423.

The cooling process 430 may include first to third cooling processes 431 to 433. FIG. 4D illustrates that the cooling process 430 includes first to third cooling processes 431 to 433, but a portion among the first to third cooling processes 431 to 433 may be omitted or the cooling process 430 may further include an additional cooling process other than the first to third cooling processes 431 to 433. The first to third cooling processes 431 to 433 may be divided according to a temperature. For example, the second cooling process 432 may be performed at a lower temperature than the first cooling process 431, and the third cooling process 433 may be performed at a lower temperature than the second cooling process 432. As an example of the inventive concept, the cooling process 430 is performed in a manner where the process temperature is gradually decreased in the first cooling process 431 to the third cooling process 433, but is not limited thereto. For example, the next cooling process may be performed at a temperature higher than that in the previous cooling process.

The first to third cooling processes 431 to 433 may be respectively performed at the first to third cooling process temperatures. The first to third cooling process temperatures may include first to third upper mold cooling temperatures 431-T1 to 433-T1 and first to third lower mold cooling temperatures 431-T2 to 433-T2. The first to third upper mold cooling temperatures 431-T1 to 433-T1 respectively indicate the temperatures of the upper mold 210 and the first to third lower mold cooling temperatures 431-T2 to 433-T2 respectively indicate the temperatures of the lower mold 220. Accordingly, the first to third cooling processes 431 to 433 may be respectively performed in states where the upper mold 210 is cooled to the first to third upper mold cooling temperatures 431-T1 to 433-T1 and the lower mold 220 is cooled to the first to third lower mold cooling temperatures 431-T2 to 433-T2.

The first information may further include information about the first to fifth upper mold cooling temperatures 431-T1 to 433-T1 and information about the first to fifth lower mold cooling temperatures 431-T2 to 433-T2.

The first information in the prediction input data 12 may include information about the first to third upper mold cooling temperatures 431-T1 to 433-T1 and information about the first to third lower mold cooling temperature 431-T2 to 433-T2.

The first information may further include information about a process time taken in the manufacturing process of the cover window CG. For example, the process time may indicate how long it takes to complete the manufacturing process.

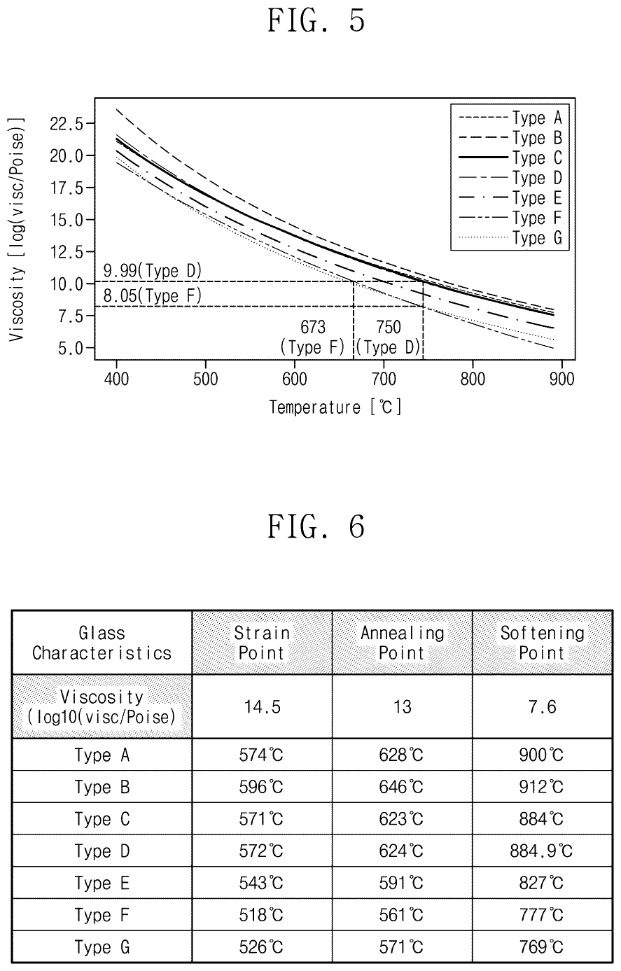

FIG. 5 is a graph showing the relationship between a temperature and a viscosity according to an embodiment of the inventive concept. The material of the window sheet material 300 (see FIG. 3B) may be one of A-type to G-type (TypeA to TypeG). In the graph of FIG. 5, the x axis indicates a temperature, and the y axis indicates the viscosity of the window sheet material 300 according to the temperature for each material type of the window sheet material 300.

Referring to FIG. 5, viscosity data of the window sheet material 300 may be calculated by the material and temperature of the window sheet material 300. A process for calculating the viscosity data may be performed in the input unit 110 (see FIG. 1A).

The window sheet material 300 may be a glass material, and its characteristics, such as a glass transition temperature, may vary according to the material of the window sheet material 300. The window sheet material 300 may include various material types. When different sample input data 11 for each of a plurality of materials is input to the learning unit 121, the number of pieces of the sample input data 11 input to the learning unit 121 may increase, and thus the training time of the learning unit 121 may become longer. Accordingly, when the viscosity data is calculated using the material and temperature of the window sheet material 300, one piece of sample input data 11 may be derived for each of the plurality of materials, and as a result, the number of pieces of sample input data 11 input to the learning unit 121 may decrease. As a result, the training time of the learning unit 121 may be reduced.

In addition, even when the dimensions of a cover window CG having a new material different from the pre-trained material is desired to be predicted, the dimensions of the cover window CG of the new material may be predicted using the existing model without new training.

For example, according to the graph shown in FIG. 5, when the material of the window sheet material 300 is D-type TypeD, the viscosity at temperature of about 750° C. may be about 9.99 log(visc/Poise).

FIG. 6 is a table showing viscosity versus temperature for each material of window sheet material according to an embodiment of the inventive concept.

Referring to FIG. 6, when the window sheet material 300 (see FIG. 3B) is a glass material, the glass characteristics of the window sheet material 300 may include a strain point, an annealing point, and a softening point.

The viscosity data (f[Poise]) may be calculated by the following Equation (1). Equation (1) may be referred to as the Voget-Fulcher-Tammann (VFT) equation, and be represented as a function of temperature (T[° C.]). In Equation 1, each of Avft, Bvft, and TO may be a unique constant value different for each material of the window sheet material 300. Avft may be differentiated according to the material of the window sheet material, and a constant value indicating a base level of the viscosity. Avft may be a constant value independent of temperature. Bvft may have a different value according to the material of the window sheet material and be a constant indicating the characteristics related to temperature. TO may indicate a virtual temperature at which the viscosity is predicted to increase to infinity. T may indicate the temperature of a material, and indicate the temperature of the window sheet material 300 in the manufacturing process of the cover window CG. The temperature of the window sheet material 300 may be provided by the upper mold 210 or the lower mold 220 in the manufacturing process.

$$log η = {A}_{VFT} + \frac{{B}_{VFT}}{T - {T}_{0}} \qquad Equation ( 1 )$$

The unique constant values may be calculated by the strain point, the annealing point, and the softening point. For example, when the glass material is A-type TypeA, the strain point is about 574° C., the annealing point is about 628° C., and the softening point is about 900° C. Furthermore, when the glass material is A-type TypeA, the viscosity at about 574° C. is about 14.5 log(visc/Poise), the viscosity at about 628° C. is about 13 log(visc/Poise), and the viscosity at about 900° C. is about 7.6 log(visc/Poise). Accordingly, the unique constant values Avft, Bvft, and TO of the window sheet material 300 may be calculated with the VFT equation as the viscosities according to three temperatures. As a result, the values of Avft, Bvft, and TO of the window sheet material 300 are predetermined, and thus the viscosity data according to the temperature for each material of the window sheet material 300 may be calculated by Equation 1.

Referring to FIGS. 4A to 4D, the pre-heat process 410 may be performed in a state where the upper mold 210 is heated to the first to fifth upper mold pre-heat temperatures 411-T1 to 415-T1 and the lower mold 220 is heated to the first to fifth lower mold pre-heat temperatures 411-T2 to 415-T2. The input unit 110 may calculate the viscosity data (namely, first viscosity data) in each of the first to fifth pre-heat processes 411 to 415 using the VFT equation, the first to fifth upper mold pre-heat temperatures 411-T1 to 415-T1, and the first to fifth lower mold pre-heat temperatures 411-T2 to 415-T2. Accordingly, the first information in the input data 10 may include the first viscosity data calculated using the first to fifth highest pre-heat temperatures 411-T1 to 415-T1 and the first to fifth lowest pre-heat temperatures 411-T2 to 415-T2.

The molding process 420 may be performed in a state where the upper mold 210 is heated to the first to third upper mold molding temperatures 421-T1 to 423-T1 and the lower mold 220 is heated to the first to third lower mold molding temperatures 421-T2 to 423-T2. The input unit 110 may calculate the viscosity data (namely, second viscosity data) using the material of the window sheet material 300, the first to third upper mold molding temperatures 421-T1 to 423-T1, and the first to third lower mold molding temperatures 421-T2 to 423-T2. The first information in the input data 10 may include the second viscosity data calculated using the first to third upper mold molding temperatures 421-T1 to 423-T1 and the first to third lower mold molding temperatures 421-T2 to 423-T2.

The cooling process 430 may be performed in a state where the upper mold 210 is heated to the first to third upper mold cooling temperatures 431-T1 to 433-T1 and the lower mold 220 is heated to the first to third lower mold cooling temperatures 431-T2 to 433-T2. The input unit 110 may calculate the viscosity data (namely, third viscosity data) using the material of the window sheet material 300, the first to third upper mold cooling temperatures 431-T1 to 433-T1, and the first to third lower mold cooling temperatures 431-T2 to 433-T2. The first information in the input data 10 may include the third viscosity data calculated using the first to third upper mold cooling temperatures 431-T1 to 433-T1 and the lower mold 220 is heated to the first to third lower mold cooling temperatures 431-T2 to 433-T2.

However, the inventive concept is not limited thereto. When a portion of the first to fifth pre-heat processes 411 to 415, the first to third molding processes 421 to 423, and the first to third cooling processes 431 to 433 is omitted, the viscosity data calculated using the upper mold and lower mold temperatures in the corresponding process may be omitted from the first information in the input data 10, and when a portion of the processes is further included, the viscosity data calculated using upper mold and lower mold temperatures in the corresponding process may be further added to the first information in the input data 10.

FIG. 7A is a perspective view of a window sheet material according to an embodiment of the inventive concept. FIG. 7B is a cross-sectional view of the window sheet material cut along A-A′ of FIG. 7A.

Referring to FIGS. 7A and 7B, the window sheet material 300 may include a top surface 310, a bottom surface 320, and side surfaces 330.

The window sheet material 300 may be flat. The window sheet material 300 is illustrated as a rectangular shape having the short sides in the first direction DR1 and the long sides in the second direction DR2, but the inventive concept is not limited thereto. The window sheet material 300 may have a rectangular shape having the long sides in the first direction DR1 and the short sides in the second direction DR2, or have a square shape or the like.

Information (fourth information) about the length and width of the window sheet material 300 in the input data 10 may further include information about the whole length 340 and whole width 350 of the window sheet material 300.

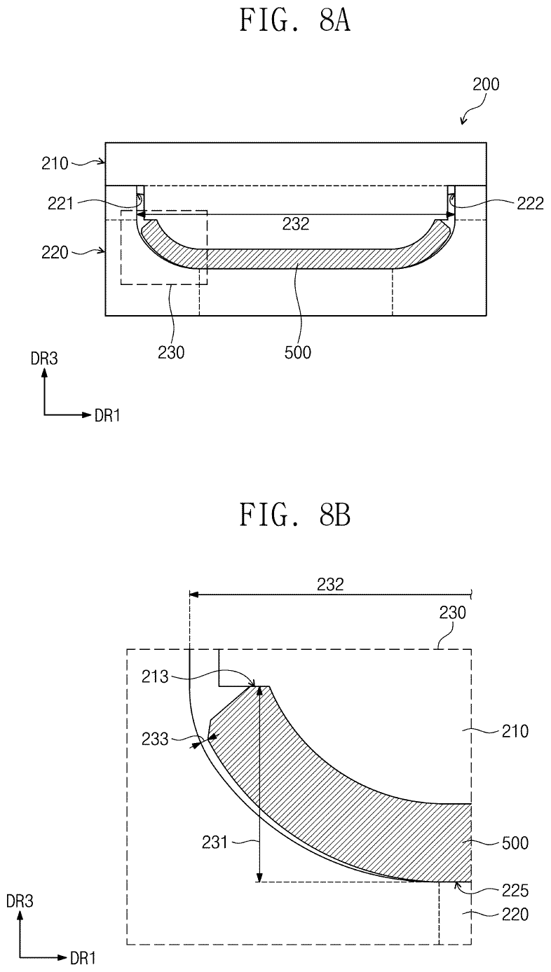

FIG. 8A is a cross-sectional view of a window molding device according to an embodiment of the inventive concept. FIG. 8B is an enlarged cross-sectional view of a partial area of FIG. 8B.

Referring to FIGS. 8A and 8B, a cover window sample 500 may be interposed between the upper mold 210 and the lower mold 220 of the window molding device 200.

The second information about the mold used in the manufacturing process in the input data 10 may include information about the width 232 of the molding part of the lower mold 220. The width 232 of the molding part may be the distance between a (1-1)-th lower portion 221 and a (1-2)-th lower portion 222.

The second information may further include information about an interval 231 between the upper mold 210 and the lower mold 220. The interval 231 may be defined as the distance from a third portion 225 of the lower mold 220 to a (2-1)-th portion 213 of the upper mold 210.

The second information may further include information about an interval 233 between the lower mold 220 and the cover window sample 500. The interval 233 between the lower mold 220 and the cover window sample 500 may be calculated as the vertical distance between the lower mold 220 and a specific point (for example, a first edge point 541 shown in FIG. 11) of the cover window sample 500.

FIG. 9 is a perspective view of the cover window sample shown in FIG. 8A.

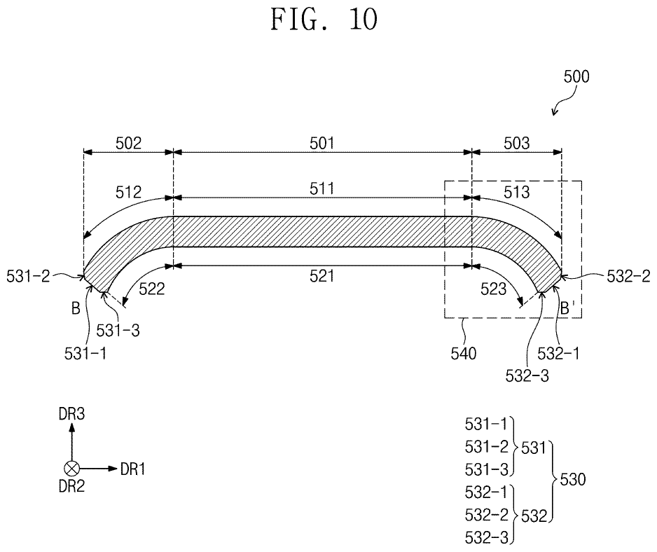

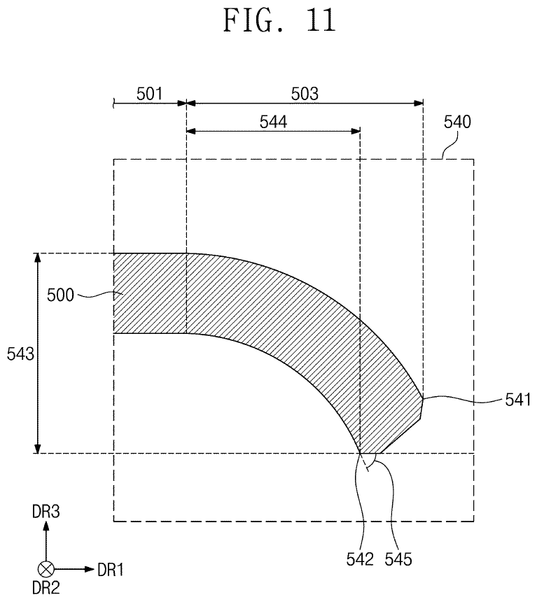

Referring to FIG. 9, the cover window sample 500 may include the top surface 510, the bottom surface 520, and the side surfaces 530 (see FIG. 10). The cover window sample 500 may have a shape in which its opposite ends are bent.

The third information about the shape of the cover window sample 500 in the input data 10 may include information about the whole length 540 and whole width 550 of the cover window sample 500.

FIG. 10 is a cross-sectional view of the cover window sample cut along B-B′ of FIG. 9.

Referring to FIGS. 9 and 10, the cover window sample 500 may include a non-bent portion 501, a first bent portion 502, and a second bent portion 503.

The top surface 510 of the cover window sample 500 may include a first top surface portion 511 positioned in the non-bent portion 501, a second top surface portion 512 positioned in the first bent portion 502, and a third top surface portion 513 positioned in the second bent portion 503.

The bottom surface 520 of the cover window sample 500 may include a first bottom surface portion 521 positioned in the non-bent portion 501, a second bottom surface portion 522 positioned in the first bent portion 502, and a third bottom surface portion 523 positioned in the second bent portion 503.

The side surfaces 530 of the cover window sample 500 may include a first side surface 531 positioned in the first bent portion 502 and a second side surface 532 positioned in the second bent portion 503.

The first side surface 531 may include a first sub-side surface 531-1, a (1-1)-th inclined surface 531-2, and a (1-2)-th inclined surface 531-3. The second side surface 532 may include a second sub-side surface 532-1, a (2-1)-th inclined surface 532-2, and a (2-2)-th inclined surface 532-3.

Since the first and second side surfaces 531 and 532 of the cover window sample 500 include the (1-1)-th to (2-2)-th inclined surfaces 531-2 to 532-3, impacts applied from the outside may be prevented from being focused on corners.

The third information in the input data 10 may further include information about the difference between the length of the top surface 510 of the cover window sample 500 and the length of the bottom surface 520 of the cover window sample 500. The length difference between the top surface 510 and the bottom surface 520 may be the difference between the length of the second top surface portion 512 in the first direction DR1 and the length of the second bottom surface portion 522 in the first direction DR1. Alternatively, the length difference between the top surface 510 and the bottom surface 520 may be the difference between the length of the third top surface portion 513 in the first direction DR1 and the length of the third bottom surface portion 523 in the first direction DR1.

FIG. 11 is an enlarged cross-sectional view of a partial area of FIG. 10.

Referring to FIGS. 10 and 11, the cover window sample 500 may include a first edge point 541 and a second edge point 542.

The first edge point 541 may be a point in which the third top surface portion 513 meets the (2-1)-th inclined surface 532-2. The second edge point 542 may be a point in which the third bottom surface portion 523 meets the (2-2)-th inclined surface 532-3.

The third information in the input data 10 may further include information about the height 543 of the cover window sample 500. The height 543 of the cover window sample 500 may be the distance from the second edge point 542 of the cover window sample 500 to the first top surface portion 511 in the third direction DR3.

The third information in the input data 10 may further include information about a bent width 544. The bent width 544 may be the distance from the boundary surface between the non-bent portion 501 and the second bent portion 503 to the second edge point 542 in the first direction DR1.

The third information in the input data 10 may further include information about each curvature of the first and second bent portions 502 and 503. The third information may further include information about each maximum curvature of the first and second bent portions 502 and 503. For example, the maximum curvature of the second bent portion 503 may be the curvature in the second edge point 542 of the cover window sample 500.

The third information in the input data 10 may further include information about each angle 545 of the first and second bent portions 502 and 503. For example, the angle 545 of the second bent portion 503 may be an angle between the (2-2)-th inclined surface 532-3 and the tangent line at the second edge point 542 of the cover window sample 500.

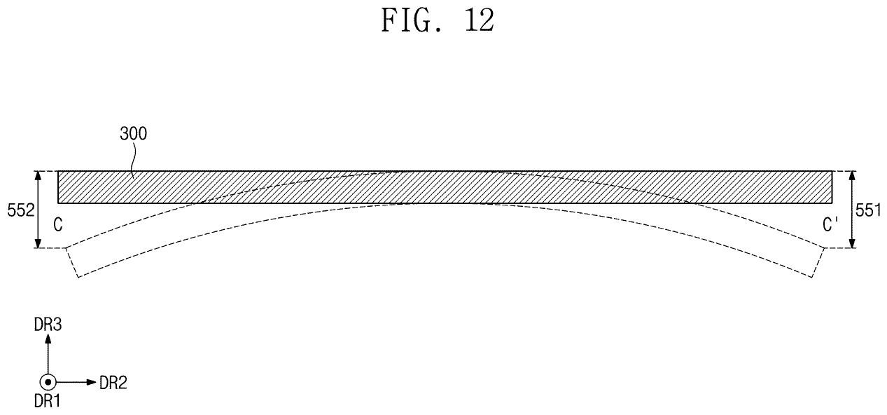

FIG. 12 is a cross-sectional view of the cover window sample cut along C-C′ of FIG. 9.

Referring to FIG. 12, the second information about a mold used in the manufacturing process in the input data 10 may further include information about the curvature correction amounts 551 and 552 of the window sheet material 300.

The window sheet material 300 may be bent in the third direction DR3 in the manufacturing process. To account for this, the window molding device 300 may proactively prevent unintended deformation by inversely applying the curvature correction amounts 551 and 552 corresponding to the expected bending of the window sheet material 300.

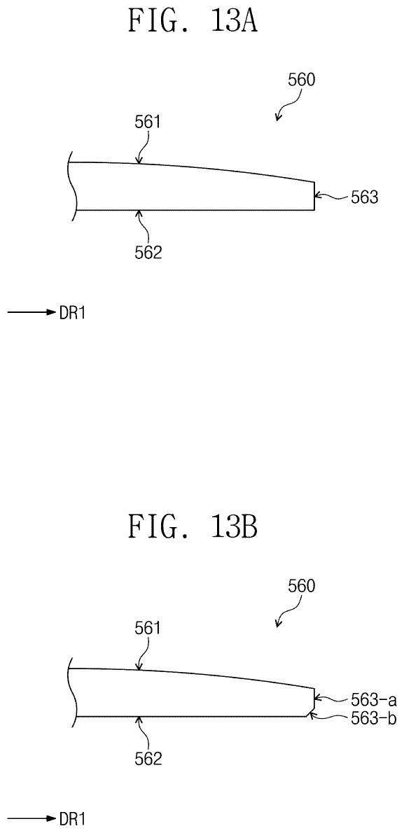

FIGS. 13A and 13B are cross-sectional views showing the shapes of cover window samples according to embodiments of the inventive concept.

Referring to FIG. 13A, the cover window sample 560 may include the top surface 561, the bottom surface 562, and the side surfaces 563. The side surfaces 563 of the cover window sample 560 may serve as a connection between the top surface 561 and the bottom surface 562. The top surface 510 of the cover window sample 560 may include a curved surface of a prescribed curvature in an edge portion, and the bottom surface 520 may have a flat shape.

The input data 10 may include information about whether one of the top surface 561 or the bottom surface 562 of the cover window sample 560 includes the curved surface of a prescribed curvature and the other includes the flat shape. For example, as shown in FIGS. 9 and 10, when the cover window sample 500 includes the curved top surface 510 and bottom surface 520, the shape of the cover window sample 500 may be referred to as having a ‘3D shape’. Like the cover window sample 560 shown in FIG. 13A or FIG. 13B, a shape in which one of the top surface 561 or the bottom surface 562 includes a curved surface and the other is flat may be referred to as having a ‘2.5D shape’. As an example of the inventive concept, information about the shapes of the cover window samples 500 and 560 in the input data 10 may be expressed as a logic “0” or logic “1” state. For example, a 3D shape sample like the cover window sample 500 may be expressed as logic “1”, and a 2.5D shape sample like the cover window sample 560 may be expressed as logic ‘0”. Besides, when the cover window samples 500 and 560 each have a different shape from the 3D shape or 2.5D shape, the input data 10 may have preset values for the shapes of the cover window samples 500 and 560.

Referring to FIG. 13B, the side surface 563 of the cover window sample 560 may further include a sub-side surface 563-a and an inclined surface 563-b. The inclined surface 563-b may connect the bottom surface 562 and the sub-side surface 563-a of the cover window sample 560.

When the side surface 563 of the cover window sample 560 further includes the inclined surface 563-b, impacts applied from the outside may be prevented from being focused on corners.

In this case, information about the shapes of the cover window samples 500 and 560 may further include information about the inclined surface 563-b.

According to the inventive concept, a learning unit (e.g., 121) may be trained by means of sample input data, and the dimensions of a cover window may be predicted using the trained learning unit (e.g., a trained model). Accordingly, without a process for actually manufacturing the cover window, the dimensions of the cover window may be predicted through the trained model and the cover window having the desired target dimensions may be manufactured.

According to an embodiment, when different sample input data for each of a plurality of materials is input to the learning unit, the number of pieces of sample input data input to the learning unit may increase, and thus the training time for the learning unit may become long. Accordingly, when viscosity data is calculated using the material and temperature of the window sheet material, one piece of sample input data may be derived from each of a plurality of materials and thus the number of pieces of sample input data input to the learning unit may decrease. As a result, the training time of the learning unit may be reduced.

While this invention has been described with reference to exemplary embodiments thereof, it should be clear to those of ordinary skill in the art that various changes and modifications may be made to the described embodiments without departing from the spirit and technical area of the invention as defined in the appended claims and their equivalents. Thus, the scope of the inventive concept should not be restricted or limited by the foregoing description.

What is claimed is:

1. A prediction and verification system comprising:
a data processing unit configured to process raw prediction data to generate processed prediction data, wherein the processed prediction data comprises viscosity data of a window sheet material, calculated based on a material of the window sheet material and a process temperature;
a machine-learning unit configured to receive the processed prediction data from the data processing unit, predict dimension values of a cover window based on the processed prediction data and output the dimension values as dimension data, wherein the dimension data includes a length, a width, and a height of the cover window; and
a determination unit configured to compare the dimension data with target data to verify suitability of the raw prediction data for use in a manufacturing process for manufacturing the cover window.

2. The prediction and verification system of claim 1, wherein the data processing unit calculates the viscosity data based additionally on first information about a condition of the manufacturing process for manufacturing the cover window, and generates the processed prediction data additionally based on second information about a mold used in the manufacturing process, third information about a shape of a cover window sample and fourth information about a length and width of the window sheet material.

3. The prediction and verification system of claim 2, wherein the first information comprises:
information about a process time of the manufacturing process;
information about the process temperature in the manufacturing process; and
information about a process pressure in the manufacturing process.

4. The prediction and verification system of claim 3, wherein the cover window is manufactured through a pre-heat process, a molding process, and a cooling process,
wherein the pre-heat process comprises a plurality of pre-heat processes,
the molding process comprises a plurality of molding processes,
the cooling process comprises a plurality of cooling processes,
the process temperature comprises temperatures in the plurality of pre-heat processes, temperatures in the plurality of molding processes, and temperatures in the plurality of cooling processes, and
the process pressure comprises one pressure in the plurality of pre-heat processes, and respective pressures in the plurality of molding processes.

5. The prediction and verification system of claim 4, wherein the viscosity data comprises:
first viscosity data calculated using the temperatures in the plurality of pre-heat processes;
second viscosity data calculated using the temperatures in the plurality of molding processes; and
third viscosity data calculated using the temperatures in the plurality of cooling processes.

6. The prediction and verification system of claim 2, wherein
the mold comprises a lower mold comprising a molding part and an upper mold disposed on the lower mold, and
the second information comprises information about:
an interval between the lower mold and the cover window sample;
a width of the molding part of the lower mold;
an interval between the upper mold and the lower mold; and
a curvature correction amount of the window sheet material.

7. The prediction and verification system of claim 6, wherein the fourth information comprises information about a length of the window sheet material and a width of the window sheet material.

8. The prediction and verification system of claim 7, wherein the fourth information further comprises information about a residual,
wherein the residual is calculated as a value obtained by subtracting one of the length or the width of the window sheet material from an average value of a length of a molding part of the upper mold and a length of the molding part of the lower mold.

9. The prediction and verification system of claim 2, wherein the third information comprises information about a difference between a length of a top surface of the cover window sample and a length of a bottom surface of the cover window sample.

10. The prediction and verification system of claim 9, wherein the third information further comprises information about a length of the cover window sample, a width of the cover window sample, and a height of the cover window sample.

11. The prediction and verification system of claim 10, wherein the third information further comprises information about:
an angle of a bent portion of the cover window sample;
a maximum curvature of the bent portion of the cover window sample;
a bent width of the bent portion; and
a curved shape of the cover window sample.

12. The prediction and verification system of claim 1, wherein the machine-learning unit comprises:
a learning unit configured to receive sample input data from a dataset to perform training in a training process to output a trained result, and output the dimension data predicted based on the processed prediction data in a prediction process; and
a comparison unit configured to receive sample dimension data from the dataset and compare the output data with the sample dimension data.

13. The prediction and verification system of claim 12, wherein the learning unit comprises:
an input layer comprising input nodes and configured to store the processed prediction data received from the data processing unit in the input nodes;
a hidden layer comprising a plurality of sub-hidden layers, wherein sub-nodes of a first sub-hidden layer among the sub-hidden layers are connected to the input nodes; and
an output layer comprising output nodes connected to sub-nodes of a last sub-hidden layer among the sub-hidden layers, and configured to output result values stored in the output nodes as the dimension data.

14. The prediction and verification system of claim 13, wherein the result values stored in the output nodes are pieces of information about the length, the width, and the height of the cover window.

15. A cover window manufacturing device comprising:
a dimension prediction system configured to predict dimension values of a cover window for generating dimension data; and
a window molding device configured to mold a window sheet material to manufacture the cover window when the dimension data matches target data,
wherein the dimension prediction system comprises:
a data processing unit configured to process raw prediction data to generate processed prediction data, wherein the processed prediction data comprises viscosity data of the window sheet material, calculated based on a material of the window sheet material and a process temperature;
a machine-learning unit configured to receive the processed prediction data from the data processing unit, predict the dimension values based on the processed prediction data and output the dimension values as the dimension data; and
a determination unit configured to compare the dimension data with the target data to verify suitability of the raw prediction data for use in a manufacturing process for manufacturing the cover window.

16. The cover window manufacturing device of claim 15, wherein the data processing unit calculates the viscosity data based additionally on first information about a condition of the manufacturing process for manufacturing the cover window, and generates the processed prediction data additionally based second information about a mold used in the manufacturing process, third information about a shape of a cover window sample, and fourth information about a length and width of the window sheet material.

17. The cover window manufacturing device of claim 16, wherein the first information comprises:
information about a process time of the manufacturing process;
information about the process temperature in the manufacturing process; and
information about a process pressure in the manufacturing process.

18. The cover window manufacturing device of claim 17, wherein the cover window is manufactured through a pre-heat process, a molding process, and a cooling process,
wherein the pre-heat process comprises a plurality of pre-heat processes,
the molding process comprises a plurality of molding processes,
the cooling process comprises a plurality of cooling processes,
the process temperature comprises temperatures in the plurality of pre-heat processes, temperatures in the plurality of molding processes, and temperatures in the plurality of cooling processes, and
the process pressure comprises one pressure in the plurality of pre-heat processes, and respective pressures in the plurality of molding processes.

19. The cover window manufacturing device of claim 18, wherein the viscosity data comprises:
first viscosity data calculated using the temperatures in the plurality of pre-heat processes;
second viscosity data calculated using the temperatures in the plurality of molding processes; and
third viscosity data calculated using the temperatures in the plurality of cooling processes.

20. A system for manufacturing a cover window, comprising:
a data processing unit configured to process raw prediction data to generate processed prediction data, wherein the processed prediction data comprises viscosity data calculated based on a material of a window sheet material and a process temperature;
a machine-learning unit configured to receive the processed prediction data from the data processing unit, predict dimension values of the cover window based on the processed prediction data and output the dimension values as dimension data, wherein the dimension data includes a length, a width, and a height of the cover window;
a determination unit configured to compare the dimension data with target data to verify suitability of the raw prediction data for use in a manufacturing process for manufacturing the cover window; and
a window molding device configured to mold the window sheet material into the cover window based on the raw prediction data when the determination unit verifies the raw prediction data.
